(19) United States
(12) Patent Application Publication
(10) Pub. No.: US 2006/0148031 A1
(43) Pub. Date: Jul. 6, 2006

(54) Hemipteran myosin light chain kinase

(76) Inventors: Ruihua Chen, Kendall Park, NJ (US); Munirathnam K Chaguturu, West Windsor, NJ (US); Debra Yuhas, Wrightstown, NJ (US); Paul Allenza, Flemington, NJ (US); Blaik P Halling, Newtown, PA (US)

Correspondence Address: John M Sheehan;FMC Corporation, 1735 Market Street, Philadelphia, PA, 19103, US

(21) Appl. No.: 10/528,631

(22) PCT Filed: Sep. 18, 2003
(86) PCT No.: PCT/US03/29901

§ 371 Date: Nov. 16, 2005

Related U.S. Application Data
(60) Provisional application No. 60/413,720, filed on Sep. 26, 2002.

(51) Int. Cl. C12N 9/12; C07H 21/04; C12P 21/06; C12N 15/74

(52) U.S. Cl. 435/69.1; 435/194; 435/320.1; 435/348; 536/23.2

(57) ABSTRACT

Novel nucleic acid sequences encoding hemipteran myosin light chain kinases are disclosed. Isolated hemipteran myosin light chain kinases are also disclosed.

### FIELD OF THE INVENTION

The present invention relates to compositions that are useful in agrochemical, veterinary or pharmaceutical fields. In particular, the invention relates to polypeptides and nucleotide sequences that encode polypeptides that are useful in the identification or development of compounds with activity as pesticides or as pharmaceuticals.

### BACKGROUND OF THE INVENTION

Myosin light chain kinase (MLCK) is believed to play an important role in regulating muscle activity. Namely, it is suspected that phosphorylation of myosin light chain (MLC) by MLCK is required for the initiation of muscle contraction, including cardiac muscle contraction (Kasturi et al., J. of Bio. Chem. (1993), Vol. 268, No. 11, pp. 7958-7964; Lei et al., J. of Bio. Chem. (1994), Vol. 269, No. 33, pp. 21078-21085; Heierhorst et al., J. of Bio. Chem. (1994), Vol. 269, No. 33, pp. 21086-21093; and Weitkamp al., J. of Bio. Chem (1998), Vol. 273, No. 31, pp. 19802-19808). Hence, interruption or inhibition of the MLCK phosphorylation of MLC can result in various biological effects (Kasturi et al., J. of Bio. Chem. (1993), Vol. 268, No. 11, pp. 7958-7964; Lei et al., J. of Bio. Chem. (1994), Vol. 269, No. 33, pp. 21078-21085; Heierhorst et al., J. of Bio. Chem. (1994), Vol. 269, No. 33, pp. 21086-21093; and Weitkamp al., J. of Bio. Chem. (1998), Vol. 273, No. 31, pp. 19802-19808). As such, there is a desire to develop ways to target this enzyme as a means of identifying biologically active compounds, including insecticides.

Twitchin, derived from unc-22 mutants of C. elegans; projectin, derived from Drosophila melanogaster; and titin, are well known MLCKs (Daley et al., J. Mol. Bio. (1998), 279, pp. 201-210; and Hardie and Hanks, The Protein Kinase FactsBook (1995), pp. 161-165). Twitchin- and projectin-like MLCKs have also been expressed and cloned from bacteria, for example E. coli, molluscs, for example, Aplysia californica, and insects, for example, Bombyx mori and Locusta migratoria (Lei et al., J. of Bio. Chem. (1994), Vol. 269, No. 33, pp. 21078-21085; Heierhorst et al., J. of Bio. Chem. (1994), Vol. 269, No. 33, pp. 21086-21093; Weitkamp al., J. of Bio. Chem. (1998), Vol. 273, No. 31, pp. 19802-19808; and NCBI Sequence Viewer, Accession Numbers AF465600, AF4656001, and AF4656002).

### SUMMARY OF THE INVENTION

The present invention relates to nucleotide sequences that encode polypeptides that are useful in the identification or development of compounds with activity as pesticides or as pharmaceuticals. The present invention also relates to polypeptide sequences that are useful in the identification or development of compounds with activity as pesticides or as pharmaceuticals. These nucleotide sequences and polypeptide sequences, will also be referred to herein as “nucleotide sequences of the invention” and “polypeptide sequences of the invention”, respectively

### DEFINITIONS

Collectively, the nucleic acids of the present invention will be referred to herein as “nucleic acids of the invention”. Also, where appropriate in the context of the further description of the invention below, the terms “nucleotide sequence of the invention” and “nucleic acid of the invention” may be considered essentially equivalent and essentially interchangeable.

Also, for the purposes of the present invention, a nucleic acid is considered to be “(in) essentially isolated (form)”—for example, from its native biological source—when it has been separated from at least one other nucleic acid molecule and sequence with which it is usually associated. Similarly, a polypeptide is considered to be “(in) essentially isolated (form)”—for example, from its native biological source—when it has been effectively separated from other polypeptide molecules with which it is normally associated with. In particular, a nucleic acid or polypeptide is considered “essentially isolated” when it has been purified at least 2-fold, in particular at least 10-fold, more in particular at least 100-fold, and up to 1000-fold or more.

### DETAILED DESCRIPTION OF THE INVENTION

The present invention relates to a nucleic acid, preferably in essentially isolated form, which nucleic acid comprises a nucleotide sequence of the invention, and in particular the nucleotide sequences of SEQ ID NO: 1. The present invention also relates to a polypeptide sequence of the invention, and in particular the polypeptide sequences of SEQ ID NO: 2 The nucleotide sequences of SEQ ID NO: 1 was derived or isolated from the Aphis gossypii organism, in the manner as further described in the Experimental Part below. The polypeptide sequence of SEQ ID NO: 2 is encoded by nucleic acid having SEQ ID NO: 1.

Generally, the nucleotide sequences of the invention, when in the form of a nucleic acid, may be DNA or RNA, and may be single stranded or double stranded. For example, the nucleotide sequences of the invention may be genomic DNA, cDNA or synthetic DNA (such as DNA with a codon usage that has been specifically adapted for expression in the intended host cell or host organism, which may for instance be designed using suitable computer programs such as Oligo-4, available from National Biosciences, Inc. (Plymouth, Minn.) and Consensus-Degenerate Hybrid Oligonucleotide Primers Software (CODEHOP) from Henikoff et al. (Nucleic Acids Research, 26, 70, 1628-1635, 1998) available on line through the Fred Hutchinson Cancer Research Center. Thus, the nucleotide sequences of the invention may contain intron sequences, and also generally comprise different splice variants.

Yet another embodiment relates to a double stranded RNA molecule directed against a nucleotide sequence of the invention (one strand of which will usually comprise at least part of a nucleotide sequence of the invention). The invention also relates to genetic constructs that can be used to provide such double stranded RNA molecules (e.g. by suitable expression in a host cell or host organism, or for example in a bacterial strain such as E. coli). For such constructs, reference is made to Maniatis et al., Molecular Cloning, a Laboratory Manual (Cold Spring Harbor Press, 1989).

In a broader sense, the term “nucleotide sequence of the invention” also comprises:
parts or fragments of the nucleotide sequences of SEQ ID NO: 1
(natural or synthetic) mutants, variants, alleles, analogs, orthologs (herein below collectively referred to as “mutants”) of the nucleotide sequences of SEQ ID NO: 1 as further described below.
parts or fragments of such (natural or synthetic) mutants;
nucleotide fusions of the nucleotide sequences of SEQ ID NO: 1 (or a part or fragment thereof) with at least one further nucleotide sequence;
nucleotide fusions of (natural or synthetic) mutants (or a part or fragment thereof) with at least one further nucleotide sequence;
   in which such mutants, parts, fragments or fusions are preferably as further described below.

The invention also comprises different splice variants of the above nucleotide sequences.

Preferably, a nucleotide sequence of the invention will have a length of at least 500 nucleotides, preferably at least 1,000 nucleotides, more preferably at least 1,500 nucleotides; and up to a length of at most 3,500 nucleotides, preferably at most 3,000 nucleotides, more preferably at most 2,600 nucleotides.

In a broader sense, the term “polypeptide sequence of the invention” also comprises:
parts or fragments of the polypeptide sequences of SEQ ID NO: 2;
(natural or synthetic) mutants, variants, alleles, analogs, orthologs (herein below collectively referred to as “mutants”) of the polypeptide sequences of SEQ ID NO: 2.
parts or fragments of such (natural or synthetic) mutants;
peptide fusions of the polypeptide sequences of SEQ ID NO: 2(or a part or fragment thereof) with at least one further polypeptide sequence;
peptide fusions of (natural or synthetic) mutants (or a part or fragment thereof) with at least one further polypeptide sequence;

Examples of parts or fragments of the nucleotide sequences of SEQ ID NO: 1 or a part or fragment of a (natural or synthetic) mutant thereof include, but are not limited to, 5′ or 3′ truncated nucleotide sequences, or sequences with an introduced in frame startcodon or stopcodon. Also, two or more such parts or fragments of one or more nucleotide sequences of the invention may be suitably combined (e.g. ligated in frame) to provide a further nucleotide sequence of the invention.

Preferably, any such parts or fragments will be such-that they comprise at least one continuous stretch of at least 100 nucleotides, preferably at least 250 nucleotides, more preferably at least 500 nucleotides, even more preferably more than 100 nucleotides, of the nucleotide sequences of SEQ ID NO: 1.

Also, it is expected that—based upon the disclosure herein—the skilled person will be able to identify, derive or isolate natural “mutants” (as mentioned above) of the nucleotide sequences of SEQ ID NO: 1. from (other individuals of) the same species (for example from an individual of a different strain or line, including but not limited to mutant strains or lines). It is also expected that—based upon the disclosure herein—the skilled person will be able to provide or derive synthetic mutants (as defmed hereinabove) of the nucleotide sequences of SEQ ID NO: 1. It is also expected that—based upon the disclosure herein—the skilled person will be able to provide or derive polypeptide having (as defined hereinabove) polypeptide sequences of SEQ ID NO: 2 by means of protein expression. In one specific embodiment, the mutant is such that it encodes the nucleotide sequence of SEQ ID NO: 1 or a part or fragment thereof.

Preferably, any mutants as described herein will have one or more, and preferably all, of the structural characteristics or conserved features referred to below for the nucleotide sequences of SEQ ID NO: 1.

Also, any mutants, parts or fragments as described herein will preferably have a degree of “sequence identity”, at the nucleotide level, with the nucleotide sequences of SEQ ID NO: 1, of at least 75%, preferably at least 80%, more preferably at least 85%, and in particular more than 90%, and up to 95% or more.

For this purpose, the percentage of “sequence identity” between a given nucleotide sequence and the nucleotide sequences of SEQ ID NO: 1, may be calculated by dividing the number of nucleotides in the given nucleotide sequence that are identical to the nucleotide at the corresponding position in the nucleotide sequences of SEQ ID NO: 1, by the total number of nucleotides in the given nucleotide sequence and multiplying by 100%, in which each deletion, insertion, substitution or addition of a nucleotide—compared to the sequences of SEQ ID NO: 1,—is considered as a difference at a single nucleotide position.

Alternatively, computer programs for determining sequence identity are publicly available. A preferred computer program for determining sequence identity is the program in Geneworks v 2.5 (Intelligenetics Inc, Mountain View Calif.), which uses a progressive alignment procedure similar to FASTA. Preferably the parameters used with the Geneworks program are: cost to open gap=50, lengthen gap=100, minimum diagonal length=4, maximum diagonal offset=125. Other computer program methods to determine identity between two sequences include, but are not limited to, GCG program package (Devereux, J., et al., Nucleic Acids Research 12(1): 387 (1984)), BLASTP, BLASTN, PASTA (Atschul, S. F. et al., J. Molec. Biol. 215: 403-410 (1990)) and VectorNTI (InforMax Inc., Bethesda, Md.). The BLAST X program is publicly available from NCBI (blast@ncbi.nlm.nih.gov) and other sources (BLAST Manual, Altschul et al., NCBI NLM NIH, Bethesda, Md. 20894; Altschul et al., J. Mol. Biol. 215: 403-410 (1990)), Vector NTI Suite Version 6 available from Informax Inc. North Bethesda, Md.

Also, in a preferred aspect, any mutants, parts or fragments as described herein will encode proteins or polypeptides having biological activity that is essentially similar to the biological activity described above for the sequences of SEQ ID NO:, i.e. to a degree of at least 50%, preferably at least 75%, and up to 90%, as measured by the assay mentioned above.

Any mutants, parts or fragments as described herein are preferably such that they are capable of hybridizing with the nucleotide sequences of SEQ ID NO: 1, i.e. under conditions of “moderate stringency”, and preferably under conditions of “high stringency”. Such conditions will be clear to the skilled person, for example from the standard handbooks, such as Sambrook et al. and Ausubel et al., mentioned above, as well as in EP 0 967 284, EP 1 085 089 or WO 00/55318.

It is also within the scope of the invention to use a fusion of a nucleotide sequence of the invention (as described above) with one or more further nucleotide sequence(s), including but not limited to one or more coding sequences, non-coding sequences or regulatory sequences. Preferably, in such fusions, the one or more further nucleotide sequences are operably connected (as described below) to the nucleotide sequence of the invention (for example so that, when the further nucleotide sequence is a coding sequence, the nucleotide fusion encodes a protein fusion as described below).

In another embodiment, the invention relates to an antisense molecule against a nucleotide sequence of the invention.

A nucleic acid, preferably in (essentially) isolated form, can be used to encode or express an amino acid sequence, for example, the amino acid sequence of SEQ D NO: 2.

On the basis of the above, and although the invention is not specifically limited to any specific explanation or mechanism, the nucleotide sequences of some embodiments of the invention encode proteins that have (biological) activity as a myosin light chain kinase. In particular, the present invention has shown activity as a myosin light chain kinase from insects of the order Hemiptera, which are aphids, leafhoppers, whiteflies, scales and true bugs that have mouthparts adapted to piercing and sucking.

As is known in the art, biological activity of this kind can be measured using standard assay techniques, for example, through competition with a labeled, known ligand for binding sites; by guanosine 5′-[γ-³⁵S]-triphosphate binding to the kinase; by fluorescent assays based on protein interactions such as fluorescence resonance energy transfer, time resolved fluorescence or fluorometic or calorimetric reporter assays; or any technology suitable for assaying MLCK proteins (see Kasturi et al.; Lei et al.; and Heierhorst et al.).

Another embodiment of the invention relates to a nucleic acid probe that is capable of hybridizing with a nucleotide sequence of the invention under conditions of moderate stringency, preferably under conditions of high stringency, and in particular under stringent conditions (all as described above). Such nucleotide probes may for instance be used for detecting or isolating a nucleotide sequence of the invention or as a primer for amplifying a nucleotide sequence of the invention; all using techniques known per se, for which reference is again made to the general handbooks such as Sambrook et al. and Ausubel et al., mentioned above.

Preferably, when to be used for detecting or isolating another nucleotide sequence of the invention, such a nucleotide probe will usually have a length of between 15 and 100 nucleotides, and preferably between 20 and 80 nucleotides. When used as a primer for amplification, such a nucleotide probe will have a length of between 25 and 75 nucleotides, and preferably between 20 and 40 nucleotides.

Generally, such probes can be designed by the skilled person starting from a nucleotide sequence of the invention - and in particular the sequences of SEQ ID NO: 1—optionally using a suitable computer algorithm. Also, as will be clear to the skilled person, such probes may be degenerate probes.

In a further aspect, the invention relates to methods for preparing mutants and genetic constructs of the nucleotide sequences of the present invention.

Natural mutants of the nucleotide sequences of the present invention may be obtained in a manner essentially analogous to the method described in the Experimental Part, or alternatively by:
construction of a DNA library from the species of interest in an appropriate expression vector system, followed by direct expression of the mutant sequence;
construction of a DNA library from the species of interest in an appropriate expression vector system, followed by screening of said library with a probe of the invention (as described below) or with a nucleotide sequence of the invention;
isolation of mRNA that encodes the mutant sequence from the species of interest, followed by cDNA synthesis using reverse transcriptase;
   or by any other suitable method(s) or technique(s) known per se, for which reference is for instance made to the standard handbooks, such as Sambrook et al., “Molecular Cloning: A Laboratory Manual” (2nd.ed.), Vols. 1-3, Cold Spring Harbor Laboratory Press (1989) and P. Ausubel et al., “Current protocols in molecular biology”, Green Publishing and Wiley Interscience, New York (1987).

Techniques for generating such synthetic sequences of the nucleotide sequences of the present invention will be clear to the skilled person and may for instance include, but are not limited to, automated DNA synthesis; site-directed mutagenesis; combining two or more parts of one or more naturally occurring sequences, introduction of mutations that lead to the expression of a truncated expression product; introduction of one or more restriction sites (e.g. to create cassettes or regions that may easily be digested or ligated using suitable restriction enzymes), and the introduction of mutations by means of a PCR reaction using one or more “mismatched” primers. These and other techniques will be clear to the skilled person, and reference is again made to the standard handbooks, such as Sambrook et al. and Ausubel et al., mentioned above.

The invention will now be further illustrated by means of the following non-limiting Experimental Part.

Experimental Part:

### EXAMPLE 1

### Myosin Light Chain Kinase Sequence Identifications

Materials and Methods.

1. Isolation of Poly(A⁺) RNA.

Cotton aphids were collected from cotton plants and placed in ice-chilled glass centrifuge tubes which had been cleaned and baked for 6 hours at 180° C. prior to use. Aliquots of approximately 0.3 gram of cotton aphids was used for isolation of poly(A) RNA and the remaining stored at −70 ° C. for future use.

In general, the RNA was isolated using QuickPrep mRNA Purification kit (Amersham Pharmacia biotech, Piscataway, N.J.) according to the manufacturer's instruction. Specifically, to a 0.3 gram aliquot of cotton aphid was added 1.5 ml of a chilled extraction buffer (contained in the QuickPrep mRNA Purificaiton kit). Upon completion of addition, the mixture was homogenized in the Braun homogenizer until a uniform suspension formed. After homogenization, 3.0 ml of elution buffer (contained in the QuickPrep mRNA Purification kit) was added. The resulting mixture was homogenized briefly in the Braun homogenizer and then centrifuged at ambient temperature at approximately 12000×g for ten minutes. The resulting supernatant was collected, applied to the resin of a oligo(dT)-cellulose spun column (contained in the QuickPrep mRNA Purificaiton kit), and washed with high salt and low salt buffers (contained in the QuickPrep mRNA Purification kit). The bound poly(A⁺) RNA was eluted with three 0.25 ml portions of elution buffer pre-warmed to 65° C. To 0.5 mL of the elute was added 50 ml of an aqueous potassium acetate solution, 10 ml of a glycogen solution, and 1 ml of 95% ethanol. The resulting mixture was stored at −20° C. for one hour and then centrifuged at maximum speed. The resulting precipitated poly(A⁺) RNA was collected and then dissolved in 50 ml of the aqueous 0.1% DEPC solution. The resulting solution was stored at −80° C. until use.

2. Design of Primers.

Oligo (version 6.0) software was used to design primers based on sequence information from FMC's proprietary Aphis gossypii Expressed Sequencing Tag (“EST”) library. Bioinformatics research indicated that two sequences in the library were the partial transcripts of target gene and flanked the region of interest, including the catalytic domain of MLCK Two pairs of primers were selected and their sequences are listed in the following:

(SEQ ID NO: 3)

| | |
|---|---|
| sense primer F1: | AGTGGACAGCGCAATTATCA, |

(SEQ ID NO: 4)

| | |
|---|---|
| antisense primer R1: | GTTACCGTAGCCGATGTCAGA, |

(SEQ ID NO: 5)

| | |
|---|---|
| sense primer F2: | GAATTTAGAGTGTACGCTGAA, |

(SEQ ID NO: 6)

| | |
|---|---|
| antisense primer R2: | TACTGGAGGCAAGTCGTTTC. |

3. Reverse Transcription and PCR Amplification (RT-PCR).

Generally, RT-PCR was done using the Thermoscript™ RT-PCR system (Invitrogen, Carlsbad, Calif.). Specifically, to a 20 μl reaction vessel was added 0.3 μg of cotton aphid poly(A⁺) RNA, 1 μl of Thermoscript™ reverse transcriptase, 1 μl of random hexamer (50 ng/ul), 4 μl 5× cDNA Synthesis Buffer, 1 μl RNaseOUT™, 1 μl 0.1 M DTT (all contained in the Thermoscript™ RT-PCR system) and appropriate volume of DEPC-treated water to a final volume of 20 μl. Upon completion of addition, the reverse transcription reaction was placed on a Geneamp® 9600 thermal cycler (available from Perkin-Elmer-ABI, Foster City, Calif.) and held at 25° C. for ten minutes. After this time, the reaction was heated to 50° C. where it was held for 50 minutes. Upon completion of this period, the reverse transcription reaction was incubated at 85° C. for 5 minutes to terminate the reaction. The reaction was stored at −20° C. or used immediately for PCR reaction.

To set up PCR reactions, 2 μl of the template solutions (reverse transcription reaction or the resultant solution from first PCR) was transferred to a 50 μl reaction vessel, and 5 μl of 10× PCR buffer, 1.5 μl of 50 mM MgCl₂, 1 μl of 10 mM dNTP, 0.4 μl of Platinum Tag DNA polymerase (5 U/μl) (all contained in the Thermoscript™ RT-PCR system), 1 μl of 10 μM sense primer, 1 μl of 10 μM antisense primer, and 38.1 μl of DEPC-treated water were added. The reaction was placed on a Perkin Elmer cycler (available from Perkin Elmer—ABI] and PCR amplifications were carried out using appropriate cycling condition depending on respective primer pairs.

The first round of PCR using reverse transcription reaction as template, as well as F1 and R1 as primer pairs, was run using the following conditions: the reaction was first heated at 94° C. for 5 min, then followed by 30 cycles of the PCR reaction: denaturing at 94° C. for 45 seconds, annealing at 54.3° C. for 45 seconds, and extension at 72° C. for 3 minutes. The completion of cycling was followed by incubation at 72° C. for 10 minutes. The resulting PCR products were characterized by agarose gel electrophoresis. At this time no PCR product band or only vague PCR product band was observed.

To enrich PCR product, secondary “nested” amplifications were carried out using 2 μl of the above PCR reaction as template, as well as F2 and R2 as primer pairs. The PCR condition is identical to that of first round except annealing at 52.3° C. PCR products were characterized by agarose gel electrophoresis. A PCR fragment of approximately 2.5 kb was excised from the gel and purified using MinElute™ Gel Extraction Kit (Qiagen Inc., 28159 Avenue Stanford, Valencia, Calif.) and the resulting DNA was ligated into a cloning vector as described below.

4. Subcloning of RT-PCR Product and Sequencing.

PCR product was ligated into pCR II TOPO vector according to manufacturer's instruction. The resulting plasmid was sequenced using T7 or SP6 as sequencing primers.

1. An isolated nucleic acid molecule having a nucleic acid sequence selected from the group consisting of: a nucleic acid sequence that encodes a polypeptide sequence of SEQ ID NO: 2, and a fragment thereof having at least 10 nucleotides.

2. The nucleic acid molecule of claim 1 consisting of SEQ ID NO: 1 or a fragment thereof having at least 10 nucleotides.

3. The nucleic acid molecule of claim 2 consisting of SEQ ID NO: 1.

4. The nucleic acid molecule of claim 1 consisting of a fragment of SEQ ID NO: 1 having at least 10 nucleotides.

5. The nucleic acid molecule of claim 1 consisting of a fragment of SEQ ID NO: 1 having 12-150 nucleotides.

6. The nucleic acid molecule of claim 1 consisting of a fragment of SEQ ID NO: 1 having 15-50 nucleotides.

7. A recombinant vector comprising a nucleic acid molecule of claim 1.

8. An isolated polypeptide molecule consisting of SEQ ID NO: 2 or a fragment thereof having at least 10 amino acids.

9. The polypeptide molecule of claim 8 consisting of SEQ ID NO: 2.

10. The polypeptide molecule of claim 8 consisting of a fragment of SEQ ID NO: 2 having at least 10 amino acids.

11. The polypeptide molecule of claim 8 consisting of a fragment of SEQ ID NO: 2 having 12-150 amino acids.

12. The polypeptide molecule of claim 8 consisting of a fragment of SEQ ID NO: 2 having 15-50 amino acids.


SEQUENCE LISTING (6 sequences)

SEQ ID NO: 1 - DNA, 2517 bp, Aphis gossypii
gaatttagagtgtacgctgaaaatgtgtatggtagaagtaaacctagtgaagtatcacctattgtaaaaacaaaagagttgttgaaaaaaccaccaaaaactaaacgttacgaagttgatgaaactggcaaaaaaattagaggaaaagcagatggcaatattaaagattacgaccaatatgtttttgacatttattcaaaatacataccacaaccagtggatattaaaacacaatccgtatatgattattatgacatattagaagaaatcggaactggtgcatttggagtagtacaccgttgtagggaacgtaaaactggaaatatttttgctgccaaatttataccagtagcacataatgttgaaaaagaattaattaaaaaagaaattgacataatgaaccaacttcatcatccgaaattgatcaatttgcatgatgctttcgaagatgaagatgaaatggtcttaatattcgaatttttgtctggaggagagctatttgaaaggatcacctcagaaggatactcaatgtccgaagcagaagtgatcaactatatgcgacagatatgtgaagctattaagcatatgcatgaaagaaatatcattcatttagatatcaaaccagaaaatataatgtgccagacaaagaagagttcaaatgtaaaactcatcgattttggattggcaacgaagttggatcctaacgaaatcgttaagatatcgacgggaactgctgagtttgcggctccagaaatagttgaaagagaaccagttggtttctatacagacatgtgggctgttggtgtcttggcatatgttcttctgagtgggctgtcaccattcgcaggagaaaacgacgtagagacgcttaaaaacgtgaaagcttgtgactgggactttgatgaagatacctttaacatagtttcagacgaaggaaaagattttatcagacgacttttgattaaaaacaaagaaaaacgaatgacagctcacgaatgtttaatacatccttggctgatgggagaccactccgatcgtacagctgcactcaactcgtccaattacacaaaaatcagagatcaaattcgcaaaaaatacagtgattgggattcgtttgctctaccacttggaagaatatcagaatacagtgctctcagaaagcttatggtagagaaatataaaatatacgaaagctcgtttgatcggcgacaagcagctccacggttcgttattaagcctcaaagtgcattctgctacgaaggacaaagtgtcaagttctactgtcgtgttattgctgtagcacaaccgacattgtcatggttccataataacgaagaattaagacaaagtgttaaattcatgaagcgatatgctggtgaagattacacgttcattatcaatagagctaagcttgatgatagaggagaatatataataagagctgaaaatacctatggctatagggaagaagtcgtattcctcaacgtacaacctttgccaaaagcagcaccggtatacagacatgaagtccaacaagtcagaagacgagaaccactcgctaatacatattatatcgaagagaaggaaagtgcaccaaatttcaccttcttattacgacctcgtgtcatacaaatacatcaaacttgcaagttactgtgctgtttaagtggcacgccaataccaactatacaatggttcagaggaaccaaagagctatctaagcgtgattacaccattacccatacggatggtgtgattacattggaaattattgactgcaagcctgaagactcaggcaaatatcgctgtgttgccactaatgtgcacggcacagacgaaaccagctgcgtagtgattgtagaaggtactgaacaaagccaagagcaggcagagttatcacataatttacttcactcagatcgcaggtacaccgatcagcatcatttcagacccgcgccaacggtcattaccaagcgcaccgccatcacttccaacgtgcacggaagcagttcggtgtcttcaaaccaatccatcaccaccaactcatcaacgaaaatcagcgacacgaccgttacctcaaccgacaggaagagcgtcaaaaaatacggcaataaactgaacgcgattggaagtccgtcaagatcccgtagttctacaaaagaattaagattatcacccgatgaagccatgtgccctccggacttttctactcgcttagtcgatacatctgtcaatgacggacagtcgctagaacttgtatgtaaagtgaccggcgatccggagccacaaatcacatggttaaagaacggaaaagctatcagttcatctaacgtcttagatctgaaatacaaaaaccgattagcgacgttgaagatcaatgaagtattccccgaagatgctggagaatatatctgcaaagctaccaactcgttgggaatgaaggaaacaagttgcaaactcacagtcaaagctgtggacgtatccaaaaacaaaagccgaaacgacttgcctccagta

SEQ ID NO: 2 - protein, 839 aa, Aphis gossypii
EFRVYAENVYGRSKPSEVSPIVKTKELLKKPPKTKRYEVDETGKKIRGKADGNIKDYDQYVFDIYSKYIPQPVDIKTQSVYDYYDILEEIGTGAFGVVHRCRERKTGNIFAAKFIPVAHNVEKELIKKEIDIMNQLHHPKLINLHDAFEDEDEMVLIFEFLSGGELFERITSEGYSMSEAEVINYMRQICEAIKHMHERNIIHLDIKPENIMCQTKKSSNVKLIDFGLATKLDPNEIVKISTGTAEFAAPEIVEREPVGFYTDMWAVGVLAYVLLSGLSPFAGENDVETLKNVKACDWDFDEDTFNIVSDEGKDFIRRLLIKNKEKRMTAHECLIHPWLMGDHSDRTAALNSSNYTKIRDQIRKKYSDWDSFALPLGRISEYSALRKLMVEKYKIYESSFDRRQAAPRFVIKPQSAFCYEGQSVKFYCRVIAVAQPTLSWFHNNEELRQSVKFMKRYAGEDYTFIINRAKLDDRGEYIIRAENTYGYREEVVFLNVQPLPKAAPVYRHEVQQVRRREPLANTYYIEEKESAPNFTFLLRPRVIQIHQTCKLLCCLSGTPIPTIQWFRGTKELSKRDYTITHTDGVITLEIIDCKPEDSGKYRCVATNVHGTDETSCVVIVEGTEQSQEQAELSHNLLHSDRRYTDQHHFRPAPTVITKRTAITSNVHGSSSVSSNQSITTNSSTKISDTTVTSTDRKSVKKYGNKLNAIGSPSRSRSSTKELRLSPDEAMCPPDFSTRLVDTSVNDGQSLELVCKVTGDPEPQITWLKNGKAISSSNVLDLKYKNRLATLKINEVFPEDAGEYICKATNSLGMKETSCKLTVKAVDVSKNKSRNDLPPV

SEQ ID NO: 3 - DNA, 20 bp, Artificial Sequence
  Synthetic Construct
agtggacagcgcaattatca

SEQ ID NO: 4 - DNA, 21 bp, Artificial Sequence
  Synthetic Construct
gttaccgtagccgatgtcaga

SEQ ID NO: 5 - DNA, 21 bp, Artificial Sequence
  Synthetic Construct
gaatttagagtgtacgctgaa

SEQ ID NO: 6 - DNA, 20 bp, Artificial Sequence
  Synthetic Construct
tactggaggcaagtcgtttc